(19) United States
(12) Patent Application Publication
(10) Pub. No.: US 2026/0008884 A1
(43) Pub. Date: Jan. 8, 2026

(54) 3D Printed Citrate-Based Scaffolds Using Additive to Improve Printability

(71) Applicant: Acuitive Technologies, Inc., Allendale, NJ (US)

(72) Inventors: Minh-Tuan Richard Tran, Ho-Ho-Kus, NJ (US); Andrew House, Elmwood Park, NJ (US)

(73) Assignee: Acuitive Technologies, Inc., Allendale, NJ (US)

(21) Appl. No.: 19/258,298

(22) Filed: Jul. 2, 2025

Related U.S. Application Data
(60) Provisional application No. 63/667,012, filed on Jul. 2, 2024.

(51) Int. Cl. C08G 63/12; A61L 27/46; A61L 27/56; A61L 27/58; B29C 64/124; B29K 67/00; B29K 309/02; B29L 31/00; B33Y 10/00; B33Y 70/10; C08G 63/78; C08K 7/00

(52) U.S. Cl. CPC C08G 63/12; A61L 27/46; A61L 27/56; A61L 27/58; B29C 64/124; B33Y 10/00; B33Y 70/10; C08G 63/78; C08K 7/00; A61L 2400/12; B29K 2067/04; B29K 2309/02; B29K 2995/006; B29L 2031/7532; C08K 2201/005; C08K 2201/011; C08K 2201/018

(57) ABSTRACT

A citrate composition is provided for use in 3D printing of end products, e.g., scaffolds. The disclosed composition incorporates water soluble salts and/or sugars into a pre-polymer to improve the viscosity for 3D printing, increase the porosity of the resulting scaffold, and improve the handling of the citrate based bioceramic compositions.

### CROSS-REFERENCE TO RELATED APPLICATIONS

The present application claims the benefit of priority to U.S. Provisional Patent Application Ser. No. 63/667,012, filed on Jul. 2, 2025, entitled “3D Printed Citrate-Based Scaffolds Using Additive to Improve Printability.” The foregoing U.S. Provisional Patent Application is incorporated herein by reference in its entirety.

BACKGROUND

### 1. Technical Field

The present disclosure is directed to citrate-based compositions for use in 3D printing highly porous scaffolds, e.g., for regenerative engineering applications.

### 2. Background Art

In recent years, 3D printing technology has made significant advances in the field of regenerative engineering, introducing new possibilities to repair multi-tissue constructs. The ability to tailor implants to individual tissues located in complex structures is crucial for optimal functionality, and 3D printing allows for the creation of patient-specific implants with intricate structures, promoting better integration with the surrounding tissues. (Wong, K. C. Orthop. Res. Rev. 2016, 8, 57-66.) For example, in regenerative engineering applications, 3D-printed scaffolds can be designed to precisely match the multifaceted requirements for tissues with stratified or gradient structures. (Zhang, B. et al. J. Mater. Chem. B, 2020, 8, 8149-8170.) Additionally, the use of biocompatible polymers and materials ensures that the implants are well-tolerated by the patient's body, minimizing the risk of rejection or adverse reactions. (Pugliese, R. et al. Annals of 3D Printed Medicine, 2021, 2, 100011)

Scaffolds for regenerative engineering applications have been fabricated using a variety of biomaterials. Notably, biodegradable elastomers have emerged as pivotal components due to their ability to match the dynamic mechanical properties of native tissue, as well as their ability to resorb into the body, avoiding the need for multiple surgeries. (Chen, S. et al. Prog. Polym. Sci. 2023, 147, 101763-101763 & Xu, C. et al. Bioact. Mater. 2022, 15, 250-271) Among biodegradable elastomers, citrate-based biomaterials hold significant promise in regenerative engineering. These materials are based on a naturally occurring metabolic molecule, which has been shown to increase stem cell energy required for tissue regeneration. (Ma, C. et al. Proc. Natl. Acad. Sci. USA., 2018, 115, E11741-E11750) In addition, citrate-based biodegradable thermoset biomaterials offer many advantages, including bulk pendant functionality to incorporate other monomers, proteins, and drugs. Moreover, the tunability of these materials allows for the precise control over scaffold properties such as mechanical strength, degradation kinetics, and porosity. (Wang, M. et al. Bioact. Mater. 2023, 19, 511-537 & Wan, L. et al. Biomacromolecules, 2023, 24, 4123-4137)

However, the printing of citrate-based biodegradable thermoset biomaterials presents a challenge due to the inherent low viscosity of the polymer. Citrate-based polymers, prior to curing (pre-polymer), are relatively low in molecular weight (<1500 Da) and viscosity. This causes sagging and other structural deformities that arise from the pre-polymer's inability to maintain its shape during the 3D printing process.

While the molecular weight of the pre-polymer can be increased with partial curing or polymer chain crosslinking, this introduces additional problems such as limited solubility in organic solvents or the need for increased solvent concentrations, which are difficult to remove. These challenges hinder the production of precise and stable structures, limiting the effectiveness of the printed polymer. Furthermore, the sagging of 3D printed fibers limits the overall size of printed parts with the inability to support taller structures.

Some methods to address these issues include employing a heated platform to partially cure the polymer during printing. However, this technique is limited as some solvents in the ink will evaporate too quickly, causing bubbles and other surface defects on printed fibers. Other methods include the use of bioceramic in the pre-polymer ink to increase viscosity. However, this also comes with drawbacks as the introduction of a bioceramic lead to reduced flexibility. Both of these methods compromise the structural integrity of the final printed scaffold.

Therefore, there is a need for biocompatible compositions that are compatible with typical 3D printing techniques. More specifically, it would be valuable for said biocompatible compositions to have both the appropriate viscosity and quick-setting properties required for efficient 3D printing without sacrificing the mechanical properties of the printed material.

### SUMMARY

The present disclosure describes a novel composition, which is beneficial for use in the 3D printing of citrate-based thermoset biomaterials. The disclosed composition boasts high porosity and structural integrity without the limitations in scaffold height or flexibility. Exemplary compositions include the incorporation of sub-micron sized water-soluble additive(s), e.g., salt(s) and/or sugar(s), into the pre-polymer ink to 1) improve the viscosity for 3D printing, 2) increase the porosity of the resulting scaffold, and 3) improve the handling of the citrate-based bioceramic scaffold. The sub-micron salt/sugar introduced to the ink acts as a thickening agent to enable the pre-polymer to maintain its shape during printing, reducing the need for excessive ceramic content and allowing the fully cured scaffold to exhibit increased flexibility. Moreover, the incorporation of the water-soluble salt/sugar serves a dual purpose as it not only increases the ink's viscosity, but also can be leached out of the fibers to enhance porosity and surface area for liquid wicking, protein binding, cell attachment, and mass transport. These improvements contribute to the overall performance and functionality of the scaffold as a biomaterial. This innovative method represents a promising advancement in overcoming the challenges associated with the 3D printing of citrate-based biodegradable thermoset biomaterials.

Additional features, functions, and benefits of the disclosed compositions and scaffolds will be apparent from the following description.

### BRIEF DESCRIPTION OF THE DRAWINGS

To assist those of skill in the art in making and using the subject matter of the present disclosure, reference is made to the appended figures, wherein:

FIG. 1 (Left) poly(octamethylene citrate) (POC) 3D printed scaffolds with 0% hydroxyapatite and 90% salt to show the flexibility of the porous scaffold, (Center) POC 3D printed scaffolds with 53% hydroxyapatite without salt showing the limited flexibility of the scaffold, and (Right) POC 3D printed scaffolds with 53% hydroxyapatite and 90% salt folded around a 2 mm rod to demonstrate the improved flexibility and handling when incorporating submicron salt into the citrate-based polymer for 3D printing.

FIG. 2 are images that demonstrate the ability of the thickening agent to hold print shape over time. Light microscope images of 3D printed scaffolds were made with 0%, 70%, 80%, and 90% salt to demonstrate the strut spreading immediately after printing for prints made with different salt concentrations, particularly lower salt concentrations.

FIG. 3 shows a SEM image of a porous citrate-based 3D printed construct where the salt has been removed, creating a highly porous structure after salt leaching and drying.

FIG. 4 is a flowchart showing steps for forming a printable composition.

FIG. 5 is a flowchart showing steps for building a 3D biodegradable scaffold.

### DESCRIPTION OF EXEMPLARY EMBODIMENTS

The present disclosure provides advantageous citrate-based compositions for use in the 3D printing of highly porous scaffolds for regenerative engineering applications.

According to exemplary embodiments, the disclosed composition comprises (a) a citrate component, (b) a multifunctional alcohol component, and (c) a thickening agent. According to further exemplary embodiments, the disclosed composition comprises (a) a citrate component, (b) a multifunctional alcohol component, (c) a particulate inorganic material, and (iv) a thickening agent.

In exemplary embodiments, the citrate component may be selected from the group consisting of citric acid, citrate, and/or an ester of citric acid. Citrate is an inherent molecule in bone anatomy and physiology, playing essential roles in regulating mineral formation and bone metabolism. In biomaterial design, citrate-based polymers present carboxylic acid and alcohol functional groups which, can be used for bioceramic interactions, conjugation sites for small molecule attachment, and as crosslinking sites to tune the mechanical and degradation properties of the resulting scaffold.

In exemplary embodiments, the multifunctional alcohol component may include a diol and/or a polyol. In exemplary embodiments, the diol may include butanediol, hexanediol, octanediol, and/or polyethylene glycerol. In exemplary embodiments, the polyol may include glycerol, beta-glycerol phosphate, and/or xylitol.

In forming the disclosed construct, the citrate component and multifunctional alcohol component (e.g., diol and/or polyol) may be reacted to form a polymer. The disclosed citrate and alcohol may be reacted, for example, at a 1.0:1.0 to 1.0:1.5 molar ratio, respectively, to form a telechelomer, i.e., a functionalized low molecular weight polymer. In exemplary embodiments, the polyol may comprise glycerol at 1-100 mol % of the total alcohol included in the composition. In other exemplary embodiments, the polyol may comprise beta-glycerol phosphate at 1-100 mol %, of the total alcohol included in the composition. Still further, the polyol may comprise xylitol at 1-100 mol %, of the total alcohol included in the composition.

The disclosed composition includes an additive/thickening agent, wherein said additive/thickening agent is a water-soluble salt and/or sugar. In exemplary embodiments, the water-soluble salt/sugar comprises at least 30% of the pre-polymer ink by mass.

Exemplary salts of the invention are represented by, but not limited to, sodium chloride, calcium chloride, sodium sulfate, potassium chloride, potassium sulfate, magnesium chloride, magnesium sulfate, sodium phosphate, potassium phosphate, sodium bicarbonate, calcium bicarbonate, and calcium sulfate.

Exemplary sugars of the invention may be simple or complex sugars. Simple sugars are the most basic forms of sugar. The sugars may be monosaccharides or disaccharides, e.g., fructose, galactose, glucose, lactose, maltose, sucrose and combinations thereof.

In exemplary embodiments, the water-soluble salt/sugar may be micro or nano-sized. In other exemplary embodiments, the water-soluble salt particles may be dissolved in water and removed through a leaching process.

The disclosed composition may further include an inorganic particulate material. In exemplary embodiments, the inorganic particulate material is introduced to form a polymer-bioceramic composite. In exemplary embodiments, the inorganic particulate materials are integrated in an amount between 0 and 60 wt. %. In exemplary embodiments, the inorganic particulate material may include one or more of hydroxyapatite, tricalcium phosphate, biphasic calcium phosphate, calcium sulfate and Bioglass (BG). BG 45S5 is one bioceramic that can be utilized according to the present disclosure. BG is composed of 43-47% silica, 22.5-26.5% calcium oxide, 5-7% phosphorus pentoxide, and 22.5-26.5% sodium oxide [Safety Data Sheet—Mo-SCI corporation; Mo-SCI Corporation. (n.d.). Retrieved May 13,2022, from mo-sci.com/wp-content/uploads/product-docs/biomaterials/GL0811-SDS.pdf] In exemplary embodiments, the bioceramic may be micro or nano-sized. In other exemplary embodiments, the bioceramic may be rod-shaped.

In exemplary embodiments, the disclosed composition forms a biodegradable polymer network.

Another embodiment of the present invention is a method for preparing a citrate-based printable composition disclosed herein comprising:
a) reacting a citrate component and multifunctional alcohol component to form a polymer;
b) adding a temporary solvent to the polymer where the solvent constitutes, for example, <60.0 wt % based on the weight of the total composition;
c) optionally adding an inorganic particulate material;
d) adding a thickening agent and mixing of the solution to fully homogenize the mixture; and
e) evaporating the excess solvent until a desired solvent concentration is reached.

The desired solvent concentration varies based on the polymer formulations, but is generally in the range of about 15-40% solvent. Qualitatively, the desired solvent concentration corresponds to when the ink can hold its shape without substantial sagging.

In exemplary embodiments, the temporary solvent is selected from dioxane, tetrahydrofuran, ethanol, or dimethylformamide.

Another embodiment of the present disclosure is a method for building a three-dimensional biodegradable scaffold, the method comprising:
a) preparing a printable composition comprising a citrate component, a multifunctional alcohol component, optionally an inorganic particulate material, a thickening agent, and a temporary solvent;
b) printing the composition to form an object representing a three-dimensional scaffold;
c) selectively curing the three-dimensional scaffold to crosslink the polymer chains;
d) removing a substantial amount of the temporary solvent from the scaffold;
e) optionally removing the thickening agent through a leaching process; and
f) optionally curing any unpolymerized polymerizable component remaining before or after step c).

In an exemplary embodiment, the printable composition has a viscosity between 40-60 Pas during extrusion. In exemplary embodiments, the printable composition comprises a temporary solvent selected from either dioxane, tetrahydrofuran, ethanol, or dimethylformamide. In certain embodiments, the temporary solvent constitutes between 7.5-40.0 wt % based on the weight of the total printable composition.

In exemplary embodiments, the composition can be printed and selectively cured to build a 3-dimensional biodegradable scaffold. In other exemplary embodiments, the disclosed three-dimensional biodegradable scaffold is a network of porous fibers. In some embodiments, the disclosed scaffolds are generally porous, e.g., 66-99% porous. The disclosed scaffold may be conformable and, in exemplary embodiments, may be cut in the operating room. In another embodiment, the disclosed scaffold may swell in liquids, e.g., the disclosed scaffold may swell in liquids by up to 300-2200%. The disclosed scaffold generally fully degrades between 1-24 months.

Exemplary compositions of the present disclosure are compatible with traditional 3D printing methods. In exemplary embodiments, scaffolds may be printed using fused deposition modeling, material extrusion, and direct ink writing. One proficient in additive manufacturing would utilize the appropriate technique and adjust the printing parameters to obtain the preferred scaffold.

### Example 1

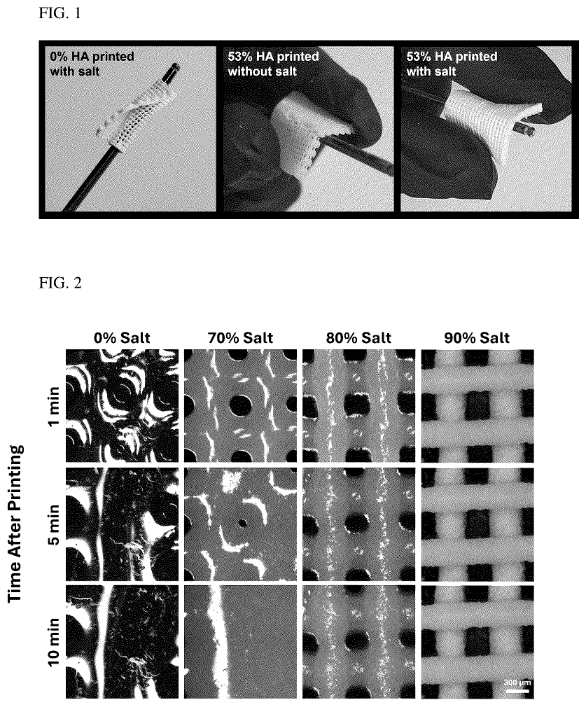

Three poly (octamethylene citrate) (POC) scaffolds were prepared following typical 3D printing methods. The first contained 53% hydroxyapatite as a bioceramic additive. The second contained 0% hydroxyapatite, but rather a water-soluble salt of the present disclosure was added. The third contained 53% hydroxyapatite as well as the water-soluble salt. Once prepared, the three scaffolds were folded around a 2 mm rod. As shown in FIG. 1, the scaffolds containing the water-soluble salt additives of the present disclosure demonstrated significantly better flexibility and malleability as it wrapped around the rod, while the scaffold containing substantial amounts of hydroxyapatite without the presence of the salt began to fracture with bending. The scaffold with both the hydroxyapatite and salt boasted a flexibility somewhere in between the two other scaffolds.

### Example 2

Four poly (octamethylene citrate) (POC) scaffolds were prepared following typical 3D printing methods. Each contained a variable amount of the water-soluble salt of the present disclosure from 0% to 90%. Once printed, the four scaffolds were imaged using a light microscope at 1 minute, 5 minutes, and 10 minutes after printing. As shown in FIG. 2, the scaffolds containing a greater percentage of the water-soluble salt additives of the present disclosure demonstrated significantly less strut spreading over time, while the scaffolds containing less salt showed increased spreading. The struts printed with 70% or less salt completely lose their initial print structure after 10 minutes.

### Example 3

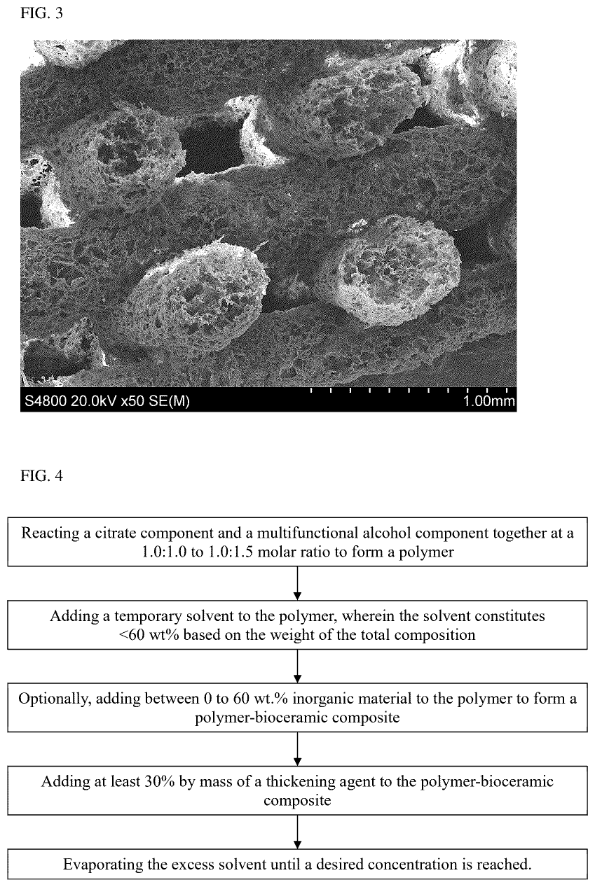

A scaffold of the present invention was prepared through 3D printing with a water-soluble salt disclosed herein. The resulting scaffold was then immersed in water to allow leaching of the water-soluble salt out of the fibers. The scaffold was then subjected to freeze drying and the resulting scaffold was observed using scanning electron microscopy (SEM). As shown in FIG. 3, the scaffold of the present invention enables the 3D printing of a citrate-based construct having substantial porosity through removal of the water-soluble salt.

### Example 4

As illustrated in FIG. 4, steps for forming a printable composition according to an embodiment of the present disclosure are shown. First, a citrate, a multifunctional alcohol component are reacted together to form a polymer. The citrate and multifunctional alcohol component (i.e., diol and/or polyol) may be reacted together at respective 1.0:1.0 to 1.0:1.5 molar ratios. Next, a temporary solvent is added to the polymer. The temporary solvent typically constitutes <60 wt % based on the weight of the total composition. Next, an inorganic particulate material is optionally combined with the polymer to form a polymer-bioceramic composite. The inorganic particulate material added may constitute between 0 to 60 wt. % of the polymer-bioceramic composite. The inorganic particulate material concentration will tune the polymer-bioceramic composite to meet the biomechanical and mineral requirements for a variety of tissue types. A thickening agent, such as a water-soluble salt, is added to the polymer-bioceramic composite to achieve a desired viscosity. The thickening agent comprising at least 30% by mass of the pre-polymer ink and will depend on the desired viscosity based on the citrate and multifunctional alcohol used and the molar ratios thereof, the wt % of the inorganic particulate material used, and the thickening agent used. Excess solvent can then be evaporated until a desired concentration and viscosity are obtained.

### Example 5

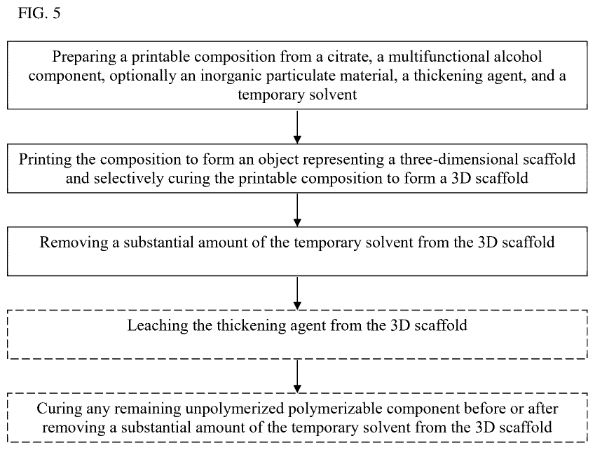

As illustrated in FIG. 5, steps for forming a 3D printed object according to an embodiment of the present disclosure are shown. First, a printable composition is prepared from a citrate component, a multifunctional alcohol component, optionally an inorganic particulate material, a thickening agent, and a temporary solvent. The printable composition may be formed utilizing the steps outlined in FIG. 4, although not limited thereto. The printable composition is then extruded and selectively cured to form a desired three-dimensional scaffold, for example a porous scaffold structure. Any known 3D printing process may be used that is suitable to form the desired shape of the 3D scaffold. Once the 3D scaffold is formed a substantial amount of the temporary solvent is removed. Optionally, the thickening agent may be leached out of the 3D scaffold which may increase the porosity of the scaffold. Further, either before or after removing a substantial amount of the temporary solvent, any remaining unpolymerized polymerizable component of the 3D scaffold may be cured.

It is appreciated that the various exemplary embodiments, and the components thereof, discussed herein may be used in combination, alternatively, and/or in addition to each other exemplary embodiment, and the components thereof.

Although the present disclosure has been described with reference to exemplary embodiments and implementations, the present disclosure is not limited by or to such exemplary embodiments/implementations.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure is not limited to the particular embodiment disclosed as the best mode contemplated for carrying out this present disclosure, but that the present disclosure will include all embodiments falling within the scope of the claims.

We claim:

1. A composition comprising (a) a citrate component, (b) a multifunctional alcohol component, and (c) a thickening agent.

2. The composition of claim 1, wherein the citrate component is at least one of citric acid, citrate, citric acid or a combination thereof.

3. The composition of claim 1, wherein the multifunctional alcohol component comprises a diol, a polyol or a combination thereof.

4. The composition of claim 3, wherein the multifunctional alcohol component comprises a diol selected from butanediol, hexanediol, octanediol, polyethylene glycerol or a combination thereof.

5. The composition of claim 3, wherein the multifunctional alcohol component comprises a polyol selected from glycerol, beta-glycerol phosphate, xylitol or a combination thereof.

6. The composition of claim 1, wherein the composition further comprises an inorganic particulate material.

7. The composition of claim 6, wherein the inorganic particulate material forms a polymer-bioceramic composite.

8. The composition of claim 6, wherein the inorganic particulate material comprises one or more of hydroxyapatite, tricalcium phosphate, biphasic calcium phosphate, calcium sulfate, and Bioglass.

9. The composition of claim 6, wherein the inorganic particulate material is present in an amount up to 60 wt.-%.

10. The composition of claim 6, wherein the inorganic particulate material is micro-sized or nano-sized.

11. The composition of claim 6, wherein the inorganic particulate material is rod-shaped.

12. The composition of claim 1, wherein the citrate component and multifunctional alcohol component define a polymer.

13. A biodegradable polymer network comprising the composition of claim 1.

14. The composition of claim 1, wherein the thickening agent is a water-soluble salt or sugar.

15. The composition of claim 14, wherein the water-soluble salt is selected from sodium chloride, calcium chloride, sodium sulfate, potassium chloride, potassium sulfate, magnesium chloride, magnesium sulfate, sodium phosphate, potassium phosphate, sodium bicarbonate, calcium bicarbonate, calcium sulfate or a combination thereof.

16. The composition of claim 14, wherein the water-soluble sugar is a monosaccharide or a disaccharide.

17. The composition of claim 16, wherein the water-soluble sugar is selected from fructose, galactose, glucose, lactose, maltose, sucrose or a combination thereof.

18. The composition of claim 14, wherein the water-soluble salt or the sugar is micro-sized or nano-sized.

19. The composition of claim 14, wherein the water-soluble salt or the sugar comprises at least 30% of a pre-polymer ink by mass.

20. A three-dimensional biodegradable scaffold, comprising the composition of claim 1.

21. The three-dimensional biodegradable scaffold of claim 20, wherein the composition comprises a network of porous fibers.

22. The three-dimensional biodegradable scaffold of claim 20, wherein the composition is 66-99% porous.

23. A method for preparing a composition, the method comprising:
a) reacting a citrate component and a multifunctional alcohol component to form a polymer;
b) adding a temporary solvent selected from either dioxane, tetrahydrofuran, ethanol, or dimethylformamide to the polymer where the solvent constitutes <60.0 wt. % based on the weight of the total composition;
c) optionally adding an inorganic particulate material;
d) adding a thickening agent and mixing of the solution to fully homogenize the mixture; and
e) evaporating excess solvent until a desired solvent concentration is reached.

24. A method for forming a three-dimensional biodegradable scaffold, the method comprising:
a) preparing a printable composition comprising a citrate component, a multifunctional alcohol component, optionally an inorganic particulate material, a thickening agent, and a temporary solvent;
b) printing the composition to form an object representing a three-dimensional scaffold;
c) selectively curing the printable composition to form an object defining a three-dimensional scaffold;
d) removing a portion of the temporary solvent from the scaffold;
e) optionally removing the thickening agent through a leaching process; and
f) optionally curing any unpolymerized polymerizable component remaining before or after step c).

25. The method of claim 24, wherein the temporary solvent is selected from dioxane, tetrahydrofuran, ethanol, or dimethylformamide, and wherein the temporary solvent comprises between 7.5 and 40.0 wt. % based on the weight of the total printable composition.

26. The method of claim 24, wherein the three-dimensional biodegradable scaffold is a network of porous fibers.

27. The method of claim 24, wherein the three-dimensional biodegradable scaffold is 66-99% porous.

28. The method of claim 24, wherein the three-dimensional biodegradable scaffold is conformable.
